(19) United States
(12) Patent Application Publication
(10) Pub. No.: US 2026/0061220 A1
(43) Pub. Date: Mar. 5, 2026

(54) SYSTEMS, APPARATUSES, AND METHODS FOR RADIATION THERAPY

(71) Applicant: Varian Medical Systems, Inc., Palo Alto, CA (US)

(72) Inventors: Joshua MCNEUR, San Jose, CA (US); Manohar BATHULA, Milpitas, CA (US)

(73) Assignee: Varian Medical Systems, Inc., Palo Alto, CA (US)

(21) Appl. No.: 18/816,316

(22) Filed: Aug. 27, 2024

(51) Int. Cl. A61N 5/10

(52) U.S. Cl. CPC A61N 5/1031

(57) ABSTRACT

A radiation treatment system includes a radiation therapy machine configured to output radiation pulses to provide a radiation dose to a patient and a beam generation and monitoring (“BGM”) board including a dose servo algorithm. The BGM board is configured to execute the dose servo algorithm in accordance with a treatment plan to control the radiation dose on a pulse-by-pulse basis according to the treatment plan.

### TECHNICAL FIELD

The present disclosure relates to systems, apparatuses, and methods for radiation therapy.

### BACKGROUND

In radiosurgery or radiotherapy (collectively referred to as radiation therapy) very intense and precisely collimated doses of radiation are delivered to a target region (volume of tumorous tissue) in the body of a patient in order to treat or destroy tumors or other lesions such as blood clots, cysts, aneurysms or inflammatory masses, for example. The goal of radiation therapy is to accurately deliver a prescribed radiation dose to the tumor/lesion and spare the surrounding healthy tissue. Ultra-high dose rate (Flash) radiotherapy delivers higher doses per pulse than conventional radiotherapy. A number of factors could impact delivery of radiation using Flash radiotherapy such as latency and variance between individual pulses of a Flash radiotherapy treatment procedure.

### SUMMARY

At least one example embodiment relates to a radiation treatment system. The radiation treatment system may include a radiation therapy machine configured to output radiation pulses to provide a radiation dose to a patient and a beam generation and monitoring (“BGM”) board including a dose servo algorithm. The BGM board may be configured to execute the dose servo algorithm in accordance with a treatment plan to control the radiation dose on a pulse-by-pulse basis according to the treatment plan.

In at least one example embodiment, the BGM board may be configured to control a first pulse of radiation output by the radiation therapy machine based on the treatment plan and to control a second pulse of radiation output by the treatment plan based on the treatment plan and the first pulse of radiation. The second pulse of radiation may be subsequent to the first pulse of radiation.

In at least one example embodiment, each pulse of radiation may be adjusted via pulse width modulation.

In at least one example embodiment, the dose servo algorithm may be a proportional integral derivative (“PID”) algorithm. In at least one example embodiment, the PID algorithm may include only a proportional term. In at least one example embodiment, the BGM board may be configured to execute the PID algorithm to adjust a pulse of radiation based on a previous pulse of the radiation.

In at least one example embodiment, the BGM board may be configured to execute the dose servo algorithm to determine a dose per pulse of radiation based on a prescribed dose of radiation. In at least one example embodiment, the BGM board may be configured to execute the dose servo algorithm to determine the dose per pulse based on the prescribed dose of radiation and a nominal dose per pulse of the radiation therapy machine. In at least one example embodiment, the nominal dose per pulse may be a percentage of a maximum dose per pulse of the radiation therapy machine. In at least one example embodiment, the percentage may be 75%.

In at least one example embodiment, the treatment plan may be a Flash radiotherapy treatment plan.

Also described herein is a method for operating a radiation treatment system. The method may include receiving a prescribed radiation dose of a treatment plan, determining a number of pulses and a dose per pulse needed to achieve the prescribed radiation dose with a dose servo algorithm, and operating a radiation therapy machine to deliver the number of pulses on a pulse-by-pulse basis.

In at least one example embodiment, the determining may determine the dose per pulse based on a nominal dose per pulse of the radiation therapy machine. In at least one example embodiment, the nominal dose per pulse may be a percentage of a maximum dose per pulse of the radiation therapy machine. In at least one example embodiment, the percentage may be 75%.

In at least one example embodiment, the treatment plan may be a Flash radiotherapy treatment plan.

In at least one example embodiment, the dose servo algorithm may be a proportional integral derivative (“PID”) algorithm. In at least one example embodiment, the PID algorithm may include only a proportional term.

In at least one example embodiment, the method may further include controlling a first pulse of radiation output by the radiation therapy machine based on the dose per pulse determined by the dose servo algorithm and controlling a second pulse of radiation output by the radiation therapy machine based on the dose per pulse determined by the dose servo algorithm and the first pulse of radiation. The second pulse of radiation may be subsequent to the first pulse of radiation.

In at least one example embodiment, the operating of the radiation therapy machine to deliver the number of pulses on the pulse-by-pulse basis may include using pulse width modulation (“PWM”) to adjust each pulse after a first pulse of radiation.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 illustrates a radiation therapy machine according to example embodiments.

FIG. 2 illustrates a radiation treatment system that includes a beam generation and monitoring (“BGM”) board and the radiation treatment system of FIG. 1 according to example embodiments.

FIG. 3 is a flow chart of a method for operating a radiation treatment system according to example embodiments.

FIG. 4 is a flow chart of step 306 of the method of FIG. 3 according to example embodiments.

### DETAILED DESCRIPTION OF EXAMPLE EMBODIMENTS

Independent of the grammatical term usage, individuals with male, female or other gender identities are included within the term.

Some detailed example embodiments are disclosed herein. However, specific structural and functional details disclosed herein are merely representative for purposes of describing some example embodiments. Example embodiments may, however, be embodied in many alternate forms and should not be construed as limited to only example embodiments set forth herein.

Accordingly, while example embodiments are capable of various modifications and alternative forms, example embodiments thereof are shown by way of example in the drawings and will herein be described in detail. It should be understood, however, that there is no intent to limit an example embodiment to the particular forms disclosed, but to the contrary, example embodiments are to cover all modifications, combinations, equivalents, and alternatives falling within the scope of an example embodiment. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or”includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, region, layer, or section discussed below could be termed a second element, region, layer, or section without departing from the teachings of example embodiment.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiment only and is not intended to be limiting of example embodiment. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, and/or elements, but do not preclude the presence or addition of one or more other features, integers, steps, operations, elements and/or groups thereof.

Example embodiments are described herein with reference to cross-sectional illustrations that are schematic illustrations of example embodiment. As such, variations from the shapes of the illustrations are to be expected. Thus, example embodiment should not be construed as limited to the shapes of regions illustrated herein but are to include deviations and variations in shapes.

When the words “about” and “substantially” are used in this specification in connection with a numerical value, it is intended that the associated numerical value include a tolerance of ±10% around the stated numerical value, unless otherwise explicitly defined. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiment belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

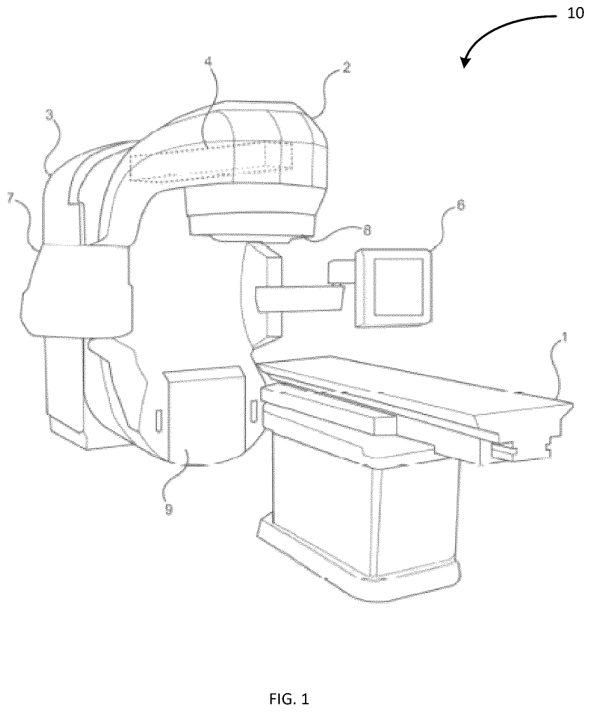

FIG. 1 illustrates a radiation therapy machine 10 according to example embodiments.

Referring to FIG. 1 a radiation therapy machine 10 includes a patient support (e.g., patient couch) 1 configured to move the patient, a gantry 2 that circles about one end of the patient couch, a stand 3 that supports the gantry 2, a beam generator 4 mounted in the gantry 2, a first imaging detector 6 mounted to the gantry 2, and a first imaging radiation source 7 mounted to the gantry 2 opposite the first imaging detector 6. The radiation therapy machine 10 may be, for example, a Halcyon (HAL) system.

The beam generator 4 generates and accelerates electrons into a beam of electrons. When a tungsten target is used, the electron beam strikes the target and generates X-rays, which are conveyed to the patient treatment area. When the tungsten target is not used, the electron beam is conveyed to the patient treatment area.

The electrons from the beam generator 4 are spatially filtered by an adjustable multi-leaf collimator (MLC) 8 having a plurality of moveable leaves of radiation-absorbent material (e.g., 120 leaves). A treatment beam of electrons or X-rays emerges from the MLC 8, and the treatment beam can have a wide range of cross-sectional patterns, as set by the positions of the leaves of the MLC 8. Prior to reaching the MLC 8, the treatment beam passes through a set of jaws, which open and close around the beam. When the jaws are closed, the treatment beam does not emerge from the collimator structure. When the jaws are open, the treatment beam emerges and strikes the patient. The first imaging detector 6 and the first imaging radiation source 7 lie in a plane that is perpendicular to the treatment beam. The treatment system may also comprise a second imaging detector 9 disposed about 6 feet away and opposite of the treatment beam (in FIG. 1, the second imaging detector 9 is shown in a folded position, folded into a compartment of the gantry 2). The second imaging detector 9 uses radiation from the treatment beam to provide an image that is perpendicular to that provided by the first imaging detector 6.

The patient support 1 need not be moveable, and may be a fixed support. The gantry 2 and the stand 3 implement one particular form of a beam-positioning mechanism that is capable of holding and/or moving the radiation beam path (e.g., trajectory) with respect to the patient. Other beam-positioning mechanisms are known to the art, and may be used in conjunction with one or more example embodiments. Beam-positioning mechanisms include, but are not limited to: gantries, ring gantries, robotic arms, beam-steering devices (including those that use electric fields and/or magnetic fields), and combinations thereof. The MLC 8 implements one particular form of a beam-shaping mechanism that is capable of modifying the cross-sectional shape of the radiation beam. Beam-shaping devices include, but are not limited to, multi-leaf collimators, iris collimators, jaw collimators, electric-field shapers (e.g., “electrostatic” shapers), magnetic-field shapers (e.g., “magnetic” lenses), and combinations thereof.

The radiation therapy machine 10 may be configured to provide ultra-high dose rate (Flash) radiotherapy. In Flash radiotherapy, dose rates exceed 40 Grays/second (Gy/s) and each pulse of radiation can deliver a dose of approximately 1 Gy. In some embodiments, delivered doses may be less than 1 Gy per pulse while maintaining a dose rate of at least 40 Gy/s.

A dose servo algorithm may be executed after a prescription including a dose per pulse and a number of pulses is determined from a radiation treatment plan in order to provide radiation therapy to a patient. In conventional radiation treatment systems, dose servo algorithms are executed by a system in communication with a radiation treatment system. For radiation treatment systems that deliver Flash radiotherapy, the dose servo algorithm should be executed with as little latency as possible. Accordingly, in one or more example embodiments described herein, the dose servo algorithm is included in a beam generation and monitoring (“BGM”) board of a radiation treatment system that includes the radiation therapy machine.

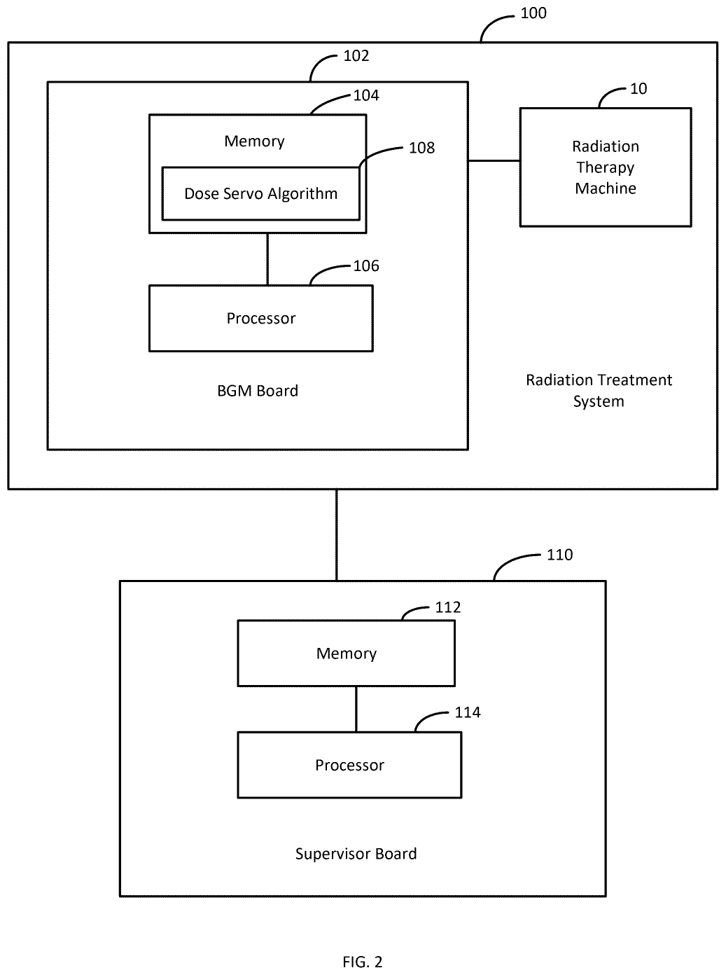

FIG. 2 illustrates a radiation treatment system 100 that includes a beam generation and monitoring (“BGM”) board 102 and the radiation therapy machine 10. In at least one example embodiment, the BGM board 102 is a system that includes at least one memory 104 and at least one processor 106. The at least one memory 104 may be configured to store instructions that may be executed by the at least one processor 106 to cause the radiation therapy machine 10 to perform one or more functions such as executing a radiotherapy procedure. In at least one example embodiment, the at least one memory 104 may include a dose servo algorithm 108. As described above, a dose servo algorithm may be configured to execute a prescription determined from a radiation treatment plan to provide radiotherapy to a patient in accordance with the radiation treatment plan. In at least one example embodiment, the radiation treatment plan may be a Flash radiotherapy treatment plan. For example, a prescription may be determined from a radiation treatment plan and may include a number of pulses of radiation to be delivered and a radiation dose per pulse. Then, the dose servo algorithm 108 may execute the prescription to deliver radiation therapy to the patient according to the radiation treatment plan.

In at least one example embodiment, the radiation treatment system 100 may be coupled to a control system such as a supervisor board 110. For example, the radiation treatment system 100 and the supervisor board 110 may be electrically coupled by a wired and/or a wireless connection. The supervisor board 110 may include at least one memory 112 and at least one processor 114. The at least one memory 112 may be configured to store instructions for controlling one or more components of the radiation therapy machine 10. The at least one processor 114 may be configured to execute the instructions stored by the at least one memory 112 to adjust and/or control one or more components of the radiation therapy machine 10. Additionally, the at least one processor 114 of the supervisor board 110 may be configured to communicate with the at least one processor 106 of the BGM board 102 in at least one example embodiment.

In at least one example embodiment, communication between the supervisor board 110 and the BGM board 102 may have a non-negligible amount of latency. For example, there may be latency of about 10 milliseconds (ms) in communications from the supervisor board 110 and the BGM board 102. During Flash radiotherapy, consecutive pulses of radiation may be delivered at pulse repetition frequencies that exceed 100 Hertz (Hz). Thus, consecutive pulses of radiation may be delivered in a time frame of 10 ms in example embodiments. Thus, communication between the supervisor board 110 and the BGM board 102 may be slower than a time between consecutive pulses of radiation and therefore, radiation pulses for Flash radiotherapy may need to be controlled by the BGM board 102 directly rather than being controlled via communication between the supervisor board 110 and the BGM board 102.

In at least one example embodiment, the processor 106 of the BGM board 102 may be configured to execute the dose servo algorithm 108 in accordance with a treatment plan to control a radiation dose on a pulse-by-pulse basis according to the treatment plan. For example, the processor 106 may be configured to control the beam generator 4 to deliver radiation pulses on a pulse-by-pulse basis according to the treatment plan. In at least one example embodiment, the processor 106 may adjust each pulse of radiation via pulse width modulation (PWM).

In at least one example embodiment, the dose servo algorithm 108 may be a proportional integral derivative (PID) algorithm. The PID algorithm may include only a proportional term such that a next pulse of radiation is dependent on a previous pulse of radiation. Thus, the processor 106 of the BGM board 102 may be configured to execute the PID algorithm to adjust a pulse of radiation based on a previous pulse of the radiation.

In at least one example embodiment, the processor 106 of the BGM board 102 may be configured to determine a dose per pulse of radiation based on a prescribed dose of radiation. For example, if a radiation treatment plan indicates a total radiation dose to be delivered to a patient, the processor 106 of the BGM board 102 may determine a total number of pulses to deliver to a patient and a dose per pulse to ensure that the total radiation dose is delivered to the patient during treatment.

In at least one example embodiment, the processor 106 of the BGM board 102 may be configured to determine the dose per pulse based on the prescribed dose of radiation and a nominal dose per pulse of the radiation therapy machine. The nominal dose per pulse may be a percentage of a maximum dose per pulse of the radiation therapy machine 10. For example, if the maximum dose per pulse of the radiation therapy machine 10 is 1 Gy, then the nominal dose per pulse may be a percentage of 1 Gy. Thus, if the percentage is 75%, the nominal dose per pulse may be 0.75 Gy. The percentage is not limited herein and may be greater than or less than 75%. The processor 106 of the BGM board 102 may determine that the dose per pulse should not exceed 0.75 Gy and may determine a dose per pulse based on this threshold and the prescribed dose of radiation from a treatment plan. The maximum dose per pulse of the radiation therapy machine 10 and the percentage to determine the nominal dose per pulse may be higher or lower than the examples described herein and should not be limited by the examples described herein.

In at least one example embodiment, the processor 106 of the BGM board 102 may be configured to determine the dose per pulse based on the prescribed dose of radiation and a nominal dose per pulse of the radiation therapy machine 10 such that the dose delivered by the radiation therapy machine 10 may be adjusted based on a previous dose. For example, if the processor 106 of the BGM board 102 determines that there should be ten pulses delivered at 0.7 Gy per pulse, the processor 106 of the BGM board 102 may send a signal to the radiation therapy machine 10 to deliver a first pulse at 0.7 Gy. However, if the processor 106 of the BGM board 102 receives a signal from the radiation therapy machine 10 that only 0.65 Gy was delivered with the first pulse, the processor 106 of the BGM board 102 may instruct the radiation therapy machine 10 to deliver the second pulse with 0.75 Gy.

Thus, radiation delivered by each pulse is adjusted based on a previous pulse to ensure that the total amount of radiation from the radiation treatment plan is delivered to the patient. The BGM board 102 is configured to make adjustments to radiation therapy in real time to course correct should there be any adjustments that need to be made to ensure that an accurate amount of radiation is delivered to a patient. The nominal dose per pulse is calculated such that there is a buffer for this correction. The BGM board 102 may be able to adjust a next pulse up to the maximum dose per pulse of the radiation therapy machine 10 due to the nominal dose per pulse being set to a value less than the maximum dose per pulse.

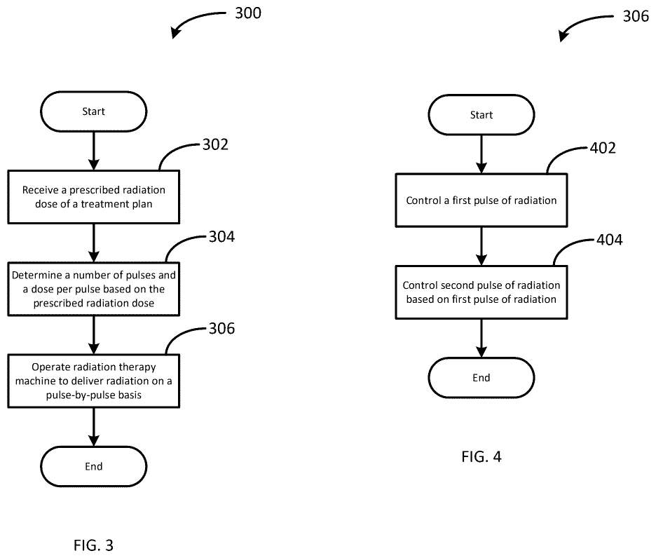

FIG. 3 is a flow chart of an example embodiment of a method 300 of operating a radiation treatment system. The method 300 will be described with respect to the radiation treatment system 100 of FIG. 2. However, example embodiments should not be limited to this example.

Referring to FIG. 3, the method 300 starts at step 302 when the processor 106 of the BGM board 102 receives a prescribed radiation dose of a treatment plan. In at least one example embodiment, the processor 106 of the BGM board 102 may receive the treatment plan from a user or a medical provider and may be configured to determine the prescribed radiation dose from the treatment plan. Alternatively or additionally, the prescribed radiation dose may be directly received by the processor 106 of the BGM board 102. In another example, the treatment plan may be obtained from a memory (not shown).

After the prescribed radiation dose is received, at step 304 the processor 106 of the BGM board 102 may determine a number of pulses and a dose per pulse in order to deliver the prescribed radiation dose. As described above, the processor 106 of the BGM board 102 may be configured to determine the dose per pulse based on the prescribed dose of radiation and a nominal dose per pulse of the radiation therapy machine 10. The nominal dose per pulse may be a percentage of a maximum dose per pulse of the radiation therapy machine 10. The dose per pulse may be determined by the processor 106 of the BGM board 102 as described above with reference to FIG. 2.

Once the number of pulses and the dose per pulse are determined, at step 306 the processor 106 of the BGM board 102 may operate the radiation therapy machine 10 to deliver the number of pulses on a pulse-by-pulse basis. Additional details of the operation of the radiation therapy machine 10, according to one or more example embodiments, are described in FIG. 4.

FIG. 4 is a flow chart of the step 306 of the method 300. The step 306 will be described with respect to the radiation treatment system 100 of FIG. 2. However, example embodiments should not be limited to this example. Moreover, although example embodiments may be described herein with regard to an initial pulse of radiation, example embodiments should not be limited to this example discussion.

Referring to FIG. 4, at step 402, the processor 106 of the BGM board 102 may control a first (e.g., an initial) pulse of radiation output by the radiation therapy machine 10. The first pulse of radiation output by the radiation therapy machine 10 may be determined by the dose servo algorithm based on the prescribed dose of radiation and a nominal dose per pulse of the radiation therapy machine 10 as described above.

At step 404, the processor 106 of the BGM board 102 may control a second pulse of radiation output by the radiation therapy machine 10. The second pulse of radiation output by the radiation therapy machine 10 may be based on the dose per pulse determined by the dose servo algorithm and the first pulse of radiation. For example, as described above, if the first pulse of radiation delivered by the radiation therapy machine 10 was less than the expected dose of the first pulse (e.g., the dose sent from the processor 106 of the BGM board 102 to the radiation therapy machine 10), then the second pulse may be adjusted to account for the difference between the expected dose and the delivered dose of radiation. Thus, as described above, the processor 106 of the BGM board 102 is configured to adjust each pulse of radiation based on a previous pulse to ensure that the total amount of radiation from the radiation treatment plan is delivered to the patient. Thus, the BGM board 102 is configured to make adjustments to radiation therapy in real time to course correct should there be any adjustments that need to be made to ensure that an accurate amount of radiation is delivered to a patient.

The processor 106 of the BGM board 102 may be configured to adjust each pulse on a pulse-by-pulse basis throughout the duration of therapy to ensure that the radiation treatment plan is carried out as accurately as possible.

The above-described systems, apparatuses, and methods provide improved radiotherapy treatment. Radiotherapy treatment as described herein is adjusted in real time to ensure that a prescribed amount of radiation is delivered by a radiation therapy machine as accurately as possible. Each pulse is adjusted to provide course correction throughout the course of the radiotherapy treatment. The systems, apparatuses, and methods described herein enable accurate delivery of Flash radiotherapy which provides an improved radiotherapy system.

Example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

We claim:

1. A radiation treatment system, the radiation treatment system comprising:
a radiation therapy machine configured to output radiation pulses to provide a radiation dose to a patient; and
a beam generation and monitoring (“BGM”) board including a dose servo algorithm, the BGM board being configured to execute the dose servo algorithm in accordance with a treatment plan to control the radiation dose on a pulse-by-pulse basis according to the treatment plan.

2. The radiation treatment system of claim 1, wherein the BGM board is configured to control a first pulse of radiation output by the radiation therapy machine based on the treatment plan, and to control a second pulse of radiation output by the treatment plan based on the treatment plan and the first pulse of radiation, wherein the second pulse of radiation is subsequent to the first pulse of radiation.

3. The radiation treatment system of claim 1, wherein each pulse of radiation is adjusted via pulse width modulation.

4. The radiation treatment system of claim 1, wherein the dose servo algorithm is a proportional integral derivative (“PID”) algorithm.

5. The radiation treatment system of claim 4, wherein the PID algorithm includes only a proportional term.

6. The radiation treatment system of claim 4, wherein the BGM board is configured to execute the PID algorithm to adjust a pulse of radiation based on a previous pulse of the radiation.

7. The radiation treatment system of claim 1, wherein the BGM board is configured to execute the dose servo algorithm to determine a dose per pulse of radiation based on a prescribed dose of radiation.

8. The radiation treatment system of claim 7, wherein the BGM board is configured to execute the dose servo algorithm to determine the dose per pulse based on the prescribed dose of radiation and a nominal dose per pulse of the radiation therapy machine.

9. The radiation treatment system of claim 8, wherein the nominal dose per pulse is a percentage of a maximum dose per pulse of the radiation therapy machine.

10. The radiation treatment system of claim 9, wherein the percentage is 75%.

11. The radiation treatment system of claim 1, wherein the treatment plan is a Flash radiotherapy treatment plan.

12. A method for operating a radiation treatment system, the method comprising:
receiving a prescribed radiation dose of a treatment plan;
determining a number of pulses and a dose per pulse needed to achieve the prescribed radiation dose with a dose servo algorithm; and
operating a radiation therapy machine to deliver the number of pulses on a pulse-by-pulse basis.

13. The method of claim 12, wherein the determining determines the dose per pulse based on a nominal dose per pulse of the radiation therapy machine.

14. The method of claim 13, wherein the nominal dose per pulse is a percentage of a maximum dose per pulse of the radiation therapy machine.

15. The method of claim 14, wherein the percentage is 75%.

16. The method of claim 12, wherein the treatment plan is a Flash radiotherapy treatment plan.

17. The method of claim 12, wherein the dose servo algorithm is a proportional integral derivative (“PID”) algorithm.

18. The method of claim 17, wherein the PID algorithm includes only a proportional term.

19. The method of claim 12, further comprising:
controlling a first pulse of radiation output by the radiation therapy machine based on the dose per pulse determined by the dose servo algorithm; and
controlling a second pulse of radiation output by the radiation therapy machine based on the dose per pulse determined by the dose servo algorithm and the first pulse of radiation, wherein the second pulse of radiation is subsequent to the first pulse of radiation.

20. The method of claim 12, wherein the operating the radiation therapy machine to deliver the number of pulses on the pulse-by-pulse basis includes using pulse width modulation (“PWM”) to adjust each pulse after a first pulse of radiation.
